SEQUENCE LISTING (20 sequences)

SEQ ID NO: 1 - protein, 10 aa, Artificial Sequence
  Artificial sequence derived from Bos taurus.
TVDMESTEVF

SEQ ID NO: 2 - protein, 12 aa, Artificial Sequence
  Artificial sequence derived from Bos taurus.
TVDMESTEVFTK

SEQ ID NO: 3 - protein, 12 aa, Artificial Sequence
  Artificial sequence derived from Bos taurus.
EQLSTSEENSKK

SEQ ID NO: 4 - protein, 14 aa, Artificial Sequence
  Artificial sequence derived from Bos taurus.
VPQLEIVPNSAEER

SEQ ID NO: 5 - protein, 15 aa, Artificial Sequence
  Artificial sequence derived from Bos taurus.
YLGEYLIVPNSAEER

SEQ ID NO: 6 - protein, 16 aa, Artificial Sequence
  Artificial sequence derived from Bos taurus.
DIGSESTEDQAMEDIK

SEQ ID NO: 7 - protein, 16 aa, Artificial Sequence
  Artificial sequence derived from Bos taurus.
YKVPQLEIVPNSAEER

SEQ ID NO: 8 - protein, 19 aa, Artificial Sequence
  Artificial sequence derived from Bos taurus.
KKYKVPQLEIVPNSAEERL

SEQ ID NO: 9 - protein, 21 aa, Artificial Sequence
  Artificial sequence derived from Bos taurus.
NTMEHVSSPSEESIISQETYK

SEQ ID NO: 10 - protein, 24 aa, Artificial Sequence
  Artificial sequence derived from Bos taurus.
QMEAEPSISSSEEIVPNPNSVEQK

SEQ ID NO: 11 - protein, 26 aa, Artificial Sequence
  Artificial sequence derived from Bos taurus.
EKVNELSKDIGSEPSTEDQAMEDIKQ

SEQ ID NO: 12 - protein, 25 aa, Artificial Sequence
  Artificial sequence derived from Bos taurus.
NANEEEYSIGSSSEESAEVATEEVK

SEQ ID NO: 13 - protein, 25 aa, Artificial Sequence
  Artificial sequence derived from Bos taurus.
NANEEEYSIGSSSEESAEVATEEVK

SEQ ID NO: 14 - protein, 15 aa, Artificial Sequence
  Artificial sequence derived from Bos taurus.
FQSEEQQQTEDELQK

SEQ ID NO: 15 - protein, 19 aa, Artificial Sequence
  Artificial sequence derived from Bos taurus.
IEKFQSEEQQQTEDELQDK

SEQ ID NO: 16 - protein, 20 aa, Artificial Sequence
  Artificial sequence derived from Bos taurus.
FQSEEQQQTEDELQDKIHPF

SEQ ID NO: 17 - protein, 25 aa, Artificial Sequence
  Artificial sequence derived from Bos taurus.
RELEELNVPGEIVESLSSSEESITR

SEQ ID NO: 18 - protein, 20 aa, Artificial Sequence
  Artificial sequence derived from Gallus gallus.
EVVGSAEAGVDAASVSEEFR

SEQ ID NO: 19 - protein, 26 aa, Artificial Sequence
  Artificial sequence derived from Gallus gallus.
FDKLPGFGDSIEAQCGTSVNVHSSLR

SEQ ID NO: 20 - protein, 13 aa, Artificial Sequence
  Synthetic sequence which is a standard tyrosine phosphorylated peptide.
RRLIEDAEYAARG

(19) United States
(12) Patent Application Publication
(10) Pub. No.: US 2009/0101581 A1
(43) Pub. Date: Apr. 23, 2009

(54) Immobilized titanium ion affinity chromatography material and its preparation and application

(76) Inventors: Hanfa Zou, Dalian (CN); Houjiang Zhou, Dalian (CN); Mingliang Ye, Dalian (CN)

Correspondence Address: SMITH, GAMBRELL & RUSSELL, 1130 CONNECTICUT AVENUE, N.W., SUITE 1130, WASHINGTON, DC, 20036, US

(21) Appl. No.: 12/149,107

(22) Filed: Apr. 25, 2008

(30) Foreign Application Priority Data
Sep. 26, 2007 (CN) 200710012966.7

(51) Int. Cl. B01D 15/08; B01J 20/281

(52) U.S. Cl. 210/656; 210/198.2

(57) ABSTRACT

The present invention relates to an immobilized titanium ion affinity chromatography material and its preparation and application. Titanium ion is immobilized on solid support by taking advantage of strong interaction between titanium ions and phosphate groups on the solid support. The immobilized titanium ion affinity chromatography material is applied to enrich phosphopeptides. Phosphopeptides are isolated because they retained on the affinity material due to the strong chelating interaction between the immobilized titanium ions and the phosphate groups on the phosphopeptides.

### FIELD OF THE INVENTION

The invention relates to enrichment and isolation of phosphopetides and more particularly to an immobilized titanium ion affinity chromatography material and its preparation and applications. Titanium ion is immobilized by taking advantage of the strong interaction between the titanium ion and phosphate group terminated on phosphate group modified solid support material surface forming immobilized titanium affinity chromatography material. The immobilized titanium affinity chromatography material is applied to enrich phosphopeptides. Phosphopeptides are strongly retained on the affinity chromatography material due to the strong chelating interaction between the immobilized titanium ion and phosphate group on phosphopeptides resulting in specific enrichment and isolation from protein digests.

### BACKGROUND OF THE INVENTION

Following with accomplishment of human genome project, the life science comes into functional genome era. About 2% of the human genome is likely to code for more than 500 protein kinases and 100 protein phosphatases, which responsible for protein phosphorylation and dephosphorylation. In all eukaryotic cells, protein phosphorylation is a common and important post translational modification of protein. Reversible phosphorylation of protein has a vital role in regulating many complex biological processes such as cellular growth, division, metabolism, metabolism and cancer progression. Additionally, it has been estimated that 30% of all proteins expressed in eukaryotic cell are phosphorylated at a given time (1. Mann, M. et al, Proteomic analysis of post-translational modifications, Nat. Biotechnol, P255-261, (2003); 2. Loyet, K. M. et al, Mass Spectrometric Contributions to the Practice of Phosphorylation Site Mapping through 2003, Mol. Cell. Proteomics, P235-245, (2005)). The identification of phosphorylation sites is very important and is still challenge at present. Recently, mass spectrometry has been developed as one of the most important technology for identification of phosphoproteins (3. Aebersold, R. et al, Mass spectrometry-based proteomics, Nature, P198-207, (2003)). However, identification of phosphoprotein by mass spectrometry is still a huge challenge. This is because, firstly, the phosphoprotein is often low abundance in cellular proteins; secondly, phosphopeptide is very difficult to be ionized during mass spectrometric detection due to its negative charge; and thirdly, ion signals of phosphopeptides are often greatly suppressed by lots of spectral signals of non-phosphopeptides in the tryptic digest. So, a prerequisite for identification phosphoproteins is the isolation and enrichment of either phosphoproteins or phosphopeptides from complex protein digests (4. Reinders. J. et al, State-of-the-art in phosphoproteomics, Proteomics, P4052-4061, (2005); 5. McLachlin, D. T. et al, Analysis of phosphorylated proteins and peptides by mass spectrometry, Curr. Opin. Chem. Biol, P591-602, (2002)).

Isolation and enrichment of phosphopeptides from peptide mixture of tryptic digest is the crucial step for mass spectrometry analysis of protein phosphorylation. By far, immobilized metal affinity chromatography (IMAC) is widely applied to enrich and isolate phosphopeptide. The enrichment and isolation of phosphopeptides with IMAC is achieved based on the strong interaction between the phosphate groups on the phosphopeptides and the immobilized metal ions, where Fe³⁺ (6. Nushe, T. S. et al, Large-scale analysis of in vivo phosphorylated membrane proteins by immobilized metal ion affinity chromatography and mass spectrometry, Mol. Cell. Proteomics, P1234-1243, (2003); 7. Moser, K. et al, Phosphoproteomic analysis of rat liver by high capacity IMAC and LC-MS/MS, J. Proteome. Res, P98-104, (2006)) and Ga³⁺ (8. Posewitz, M. C. et al, Immobilized gallium(III) affinity chromatography of phosphopeptides, Anal. Chem, P2883-2992, (1999)) were still widely used. With IMAC technology, the chelating groups such as the iminodiacetic acids (IDA) (9. Pan, C. et al, Enrichment of phosphopeptides by Fe³⁺-immobilized mesoporous nanoparticles of MCM-41 for MALDI and nano-LC-MS/MS analysis, J. Proteome. Res, P3114-3124, (2006)) and nitrilotriacetic acid (NTA) (10. Dunn, J. D. et al, Detection of phosphopeptides using Fe(III)-nitrilotriacetate complexes immobilized on a MALDI plate, Anal. Chem, (5), P1574-1580, (2006)) are often covalent linked to chromatography media as ligands. One typical weakness of IMAC using Fe³⁺, Ga³⁺ or Ni²⁺ is that it not only binds phosphopeptides but also acidic peptides and peptides containing histidine residue, which certainly leads to reduce their specificity for enrichment of phsophopeptides (11. Ficarro, S. B, et al, Phosphoproteome analysis by mass spectrometry and its application to Saccharomyces cerevisiae, Nat. Biotechnol, P301-305, (2002)). Although one approach by blocking the acidic residues such as glutamic acid and aspartic acid using methyl esterification has been shown to reduce non-specific adsorption of non-phosphopeptides, the low yield of derivatization and the side reactions comprise sensitivity of this method and increase the complexity of samples. Additionally, metal oxides being applied to isolate and enrich phosphopeptide shown more specific than IMAC beads. (12. Kweon, H. K. et al, Selective zirconium dioxide-based enrichment of phosphorylated peptides for mass spectrometric analysis, Anal. Chem, P1743-1749, (2006); 13. Zhou, H. et al, Highly specific enrichment of phosphopeptides by zirconium dioxide nanoparticles for phosphoproteome analysis, Electrophoresis, P2201-2215, (2007)), TiO₂ (14. Cantin, G. T. et al, Optimizing TiO₂-based phosphopeptide enrichment for automated multidimensional liquid chromatography coupled to tandem mass spectrometry, Anal. Chem, P4666-4673, (2007); 15. Thingholm, T. E. et al, Highly selective enrichment of phosphorylated peptides using titanium dioxide, Nat. Protoc, P1929-1935, (2006); 16. Pocsfalvi, G. et al, Phosphorylation of B14.5α subunit from bovine heart complex I identified by titanium dioxide selective enrichment and shotgun proteomics, Mol. Cell. Proteomics, P231-237, (2007)) and Al₂O₃ (17. Wolschin, F. et al, Enrichment of phosphorylated proteins and peptides from complex mixtures using metal oxide/hydroxide affinity chromatography (MOAC), Proteomics, P4389-P4397, (2005)). The reason that why metal oxides such as ZrO₂ and TiO₂ have specific affinity for phosphopeptides is due to the strong interaction between their metal ions and phosphate groups on the phosphopeptides.

However, the disadvantage of using ZrO₂ and TiO₂ for phosphopeptide enrichment is that the binding of some phosphopeptides may be not efficient due to steric hindrance resulted from no spacer arm. If Zr⁴⁺ or Ti⁴⁺ could be immobilized onto solid support via ligands, the resulting Zr⁴⁺ or Ti⁴⁺-based IMAC should have higher enrichment efficiency for phosphopeptide by introducing a space arm. Recently, Zr⁴⁺-IMAC has been shown more specificity and selectivity than conventional IMAC (18. Zhou, H. et al, Zirconium phosphonate-modified porous silicon for highly specific capture of phosphopeptides and MALDI-TOF MS analysis, J. Proteome. Res, P2431-2437, (2006); 19. Feng, S. et al., Immobilized zirconium ion affinity chromatography for specific enrichment of phosphopeptides in phosphoproteome analysis, Mol. Cell. Proteomics, P1656-1665, (2007)).

This invention presents a Ti⁴⁺-based IMAC, the methods for preparation of Ti⁴⁺-IMAC and application of Ti⁴⁺-IMAC for enrichment of phosphopeptides. By far, there has no literature describing of Ti⁴⁺-IMAC and its preparation and applications.

### SUMMARY OF THE INVENTION

The purpose of this invention is to provide an immobilized titanium ion affinity chromatography (Ti⁴⁺-IMAC) material, and its preparation and application. The said immobilized titanium affinity chromatography material from the invention presents with high specificity and selectivity for phosphopeptide.

The technology program of the invention is as follows:

Immobilized titanium ion affinity chromatography (Ti⁴⁺-IMAC) material could be prepared by incubating the titanium ion solution with solid support containing phosphate groups. The titanium ions are immobilized on the surface of the solid support via phosphate ligands because of the strong interaction between titanium ions and phosphate groups.

Above mentioned solid support containing phosphate groups having following structure wherein phosphate groups are covalent linked on the surface of the solid support.

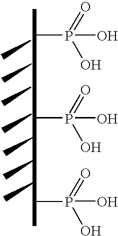

Preparation of immobilized titanium ion affinity chromatography material: incubation of solid support containing phosphate groups in the titanium ion solution, the titanium ions are immobilized on the surface of the solid support through the phosphate ligands by taking advantage of the strong interaction between titanium ions and phosphate groups, and thus the immobilized titanium affinity chromatography (Ti⁴⁺-IMAC) material is prepared.

FIG. 1a gives the schematics for the immobilization of titanium ions. The Ti⁴⁺-IMAC is obtained by placing the solid support containing phosphate groups in the titanium ion solution with stirring or simply wetting the solid support containing phosphate groups with titanium ion solution. Titanium ions are immobilized on the solid support through the strong chelating interaction between with titanium ions and phosphate groups.

Herein, the titanium solution could be titanium sulphate solution. The affinity chromatographic material containing phosphate groups could be prepared by chemical derivatization of solid support, which is achieved by covalently coupling of phosphate groups on the surface.

Herein the above-mentioned solid support could be conventional chromatographic support such as silicon particles, organic polymer beads, agrose beads, or monolithic_material, nanomaterial, mesopore material (such as mesopore MCM-41), magnetic particles, chip or other particle support.

The procedure of chemical derivatization is to modify the surface of solid support by amination and subsequent phosphoryl reaction resulting in the formation of phosphate group modified material. Other reaction such as phosphonate reaction can be also introduced. Any reactions which can generate phosphate groups on the surface of solid support could be used.

Wherein the mentioned affinity chromatography material is prepared by polymerization reaction using functional monomer containing phosphate group, the polymerization reactions could be bulk polymerization, suspension polymerization or emulsion polymerization.

Wherein the functional monomer could be ethylene glycol methacrylate phosphate (EGMP) or any other monomer containing phosphate group, the cross linker such as bis-acrylamide (BAA), divinylbenzene and ethylene dimethacrylate et al could be also used.

The application of immobilized titanium ion affinity chromatographic material: The present invention offers methods of enrichment of phosphopeptide by using immobilized titanium ion affinity chromatography (Ti⁴⁺-IMAC) as isolation material. The phosphopeptides, with phosphate group, are specifically retained on the affinity material due to the strong chelating interaction between immobilized titanium ion and phosphate group on phosphopeptides. Thus phosphopeptides could be specifically separated and isolated from the complex protein digests.

As shown in the FIG. 1b, the phosphopeptides are retained by Ti⁴⁺-IMAC due to the strong chelating interaction between the titanium ion and phosphate groups on phosphopeptides and non-phosphopeptides aren't retained which resulted in specific isolation of phosphopeptides from complex peptide mixture with the presence of large amount of non-phosphopeptides.

### ADVANTAGES OF THE INVENTION

Ti⁴⁺-IMAC presented by this invention has high specificity and selectivity for isolation of phosphopeptides. Its specificity and selectivity is higher than metal oxide such as TiO₂ and ZrO₂, and it doesn't have significant bias toward singly and multiply phosphorylated peptides.

### BRIEF DESCRIPTION OF THE DRAWINGS

FIG. 1a is the scheme for preparation of Ti⁴⁺-IMAC.

FIG. 1b is the scheme for isolation and enrichment of phosphopeptides using the Ti⁴⁺-IMAC.

FIG. 2 is the MALDI mass spectrum of the phosphopeptides enriched by Ti⁴⁺-IMAC from tryptic digests of α-casein. Wherein the sequence and the phosphorylation sites of the phosphopeptides are listed in Table.1. Ti⁴⁺-IMAC is made from polymer containing phosphate group.

FIG. 3 is the MALDI mass spectrum of the phosphopeptides enriched by polymer Ti⁴⁺-IMAC from the tryptic digests of β-casein and ovalbumin and standard tyrosine phosphorylated peptides. Wherein the sequence and the phosphorylation sites of the phosphopeptides are listed in Table.1. Ti⁴⁺-IMAC is made from polymer containing phosphate group.

FIG. 4 is the MALDI mass spectra of selective enrichment of phosphopeptides from semi-complex_peptide mixtures of tryptic digest of α-casein and BSA. Wherein (a) Direct analysis of the complex peptide mixture with molar ratio of 1:100; (b) α-casein digest after isolated and enriched by Ti⁴⁺-IMAC; (c) peptide mixtures of tryptic digests of α-casein and BSA with molar ratio of 1:100 after enriched by Ti⁴⁺-IMAC; (d) peptide mixtures of tryptic digests of α-casein and BSA with molar ratio of 1:500 after enriched by Ti⁴⁺-IMAC. Ti⁴⁺-IMAC is made from polymer containing phosphate group.

FIG. 5 is the MALDI mass spectrum of the enriched phosphopeptides by Ti⁴⁺-IMAC made from phosphate group modified inorganic MCM-41 material from tryptic digests of α-casein. Wherein the sequence and the phosphorylation sites of the phosphopeptides are listed in Table.1

FIG. 6 is the MALDI mass spectrum of the enriched phosphopeptides by Ti⁴⁺-IMAC made from phosphate group modified inorganic MCM-41 material from the tryptic digests of β-casein and ovalbumin and a standard tyrosine phosphorylated peptide. Wherein the sequence and the phosphorylation sites of the phosphopeptides are listed in Table.1.

FIG. 7 is the MALDI mass spectra of selective enrichment of phosphopeptides from the semi-complex peptide mixtures of tryptic digestions of α-casein and BSA (with mole ratio of 1:100, 1:500). Wherein (a) Direct analysis of the complex peptide mixture with molar ratio of 1:100; (b) α-casein digest after enriched by Zr⁴⁺-IMAC; (c) peptide mixtures of tryptic digestions of α-casein and BSA with molar ratio of 1:100 after enriched by Zr⁴⁺-IMAC; (d) peptide mixtures of tryptic digestions of α-casein and BSA with molar ratio of 1:500 after enriched by Zr⁴⁺-IMAC. Zr⁴⁺-IMAC is made from polymer containing phosphate group.

FIG. 8 is the MALDI mass spectra of selective enrichment of phosphopeptides from the semi-complex peptide mixtures of tryptic digestions of α-casein and BSA (with mole ratio of 1:100, 1:500) based on GMA-EDMA particle. Wherein (a) Direct analysis of the complex peptide mixture with molar ratio of 1:100; (b) α-casein digest after enriched by Zr⁴⁺-IMAC based on GMA-EDMA particle; (c) peptide mixtures of tryptic digests of α-casein and BSA with molar ratio of 1:100 after enriched by Zr⁴⁺-IMAC based on GMA-EDMA particle; (d) peptide mixtures of tryptic digests of α-casein and BSA with molar ratio of 1:500 after enriched by Zr⁴⁺-IMAC based on GMA-EDMA particle.

FIG. 9 is the MALDI mass spectra of selective enrichment of phosphopeptides from the semi-complex peptide mixtures of tryptic digestions of α-casein and BSA(with mole ratio of 1:100, 1:500) by Fe³⁺-IMAC. Wherein (a) Direct analysis of the complex peptide mixture with molar ratio of 1:100; (b) α-casein digest after enriched by Fe³⁺-IMAC; (c) peptide mixtures of tryptic digests of α-casein and BSA with molar ratio of 1:100 after enriched by Fe³⁺-IMAC; (d) peptide mixtures of tryptic digests of α-casein and BSA with molar ratio of 1:500 after enriched by Fe³⁺-IMAC.

FIG. 10 is the MALDI mass spectra of selective enrichment of phosphopeptides from the semi-complex peptide mixtures of tryptic digestions of α-casein and BSA (with mole ratio of 1:100, 1:500) by TiO₂. Wherein (a) Direct analysis of the complex peptide mixture with molar ratio of 1:100; (b) α-casein digest after enriched by TiO₂; (c) peptide mixtures of tryptic digests of α-casein and BSA with molar ratio of 1:100 after enriched by TiO₂; (d) peptide mixtures of tryptic digests of α-casein and BSA with molar ratio of 1:500 after enriched by TiO₂.

FIG. 11 is the MALDI mass spectra of selective enrichment of phosphopeptides from the semi-complex peptide mixtures of tryptic digests of α-casein and BSA (with mole ratio of 1:100, 1:500) by ZrO₂. Wherein (a) Direct analysis of the complex peptide mixture with molar ratio of 1:100; (b) α-casein digest after enriched by ZrO₂; (c) peptide mixtures of tryptic digests of α-casein and BSA with molar ratio of 1:100 after enriched by ZrO₂; (d) peptide mixtures of tryptic digestions of α-casein and BSA with molar ratio of 1:500 after enriched by ZrO₂.

### DESCRIPTION OF THE INVENTION IN DETAIL

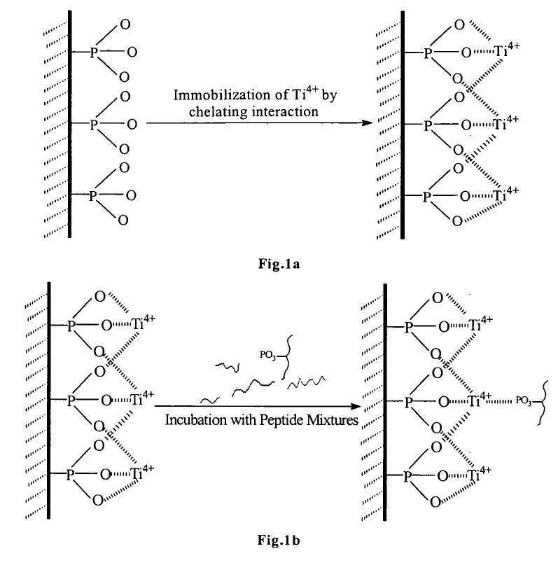

The immobilized titanium affinity chromatography (Ti⁴⁺-IMAC) material presented in this invention is prepared by immobilization of titanium ion on solid support containing phosphate groups based on the strong interaction between titanium ion and phosphate group on the surface of the solid support.

Wherein the titanium ion solution could be titanium sulphate (Ti(SO₄)₂) solution or any other solution containing titanium ion. As shown in the FIG. 1a, titanium ion is immobilized onto solid support when the phosphate group modified solid support is placed in or wetted with titanium solution, stationary or stirring for a while, which resulting in formation of the Ti⁴⁺-IMAC material.

Wherein solid support containing phosphate groups refers to solid support with surface modified by phosphate groups, i.e. with phosphate group terminated surface. Such material containing phosphate groups is mainly prepared by the following two methods:

1) Preparation of polymer solid support containing phosphate group by coupling of phosphate groups onto solid support by chemical derivatization. Wherein solid support is conventional chromatographic support such as silicon gel, polymer beads and agarose particle et al., and is monolithic material, nanomaterial, mesoporous material, magnetic beads, chip and other particle solid support. The phosphate group is coupled onto the solid support by chemical reaction with reactive group on the surface of the solid support. Herein, MCM-41 mesoporous molecule sieve is used as an example of solid support to demonstrate the preparation of phosphate group modified support in this invention. However, the invention isn't limited to mesoporous molecule sieve and any other solid support which can be derivatized with phosphate group can also be used.
2) Preparation of polymer solid support containing phosphate group by polymerization of monomer containing phosphate group. Herein, this invention gives an example with free-radical co-polymerization of EGMP and BAA as cross-linker. However, the monomer containing phosphate group isn't limited to the EGMP and other functional monomers containing phosphate group could also be used. The cross-linker isn't limited to BAA and other cross-linkers such as divinylbenzene and ethylene dimethacrylate et al could also be used.

This invention provides a new method for isolation and enrichment phosphopeptides using Ti⁴⁺-IMAC. The isolation and enrichment of phosphopeptides mainly comprises three steps of sample loading, washing and elution: peptide mixture containing phosphopeptides was firstly loaded onto Ti⁴⁺-IMAC material, and phosphopeptides are retained on the affinity material due to the strong interaction between the phosphopeptides and the immobilized Ti⁴⁺; After sample loading, Ti⁴⁺-IMAC material is subjected to wash to remove non-specific adsorption of non-phosphopeptides and then specifically retained phosphopeptides are eluted. Sample is typically loaded after the peptide mixture is acidified by acid such as formic acid, trifluoroacetic acid and acetic acid. After loading sample, organic solvent and salt are often added into washing buffer to remove non-specific adsorption peptides resulting from hydrophobic and electrostatic interaction. For example, 50% acetonitrile (ACN)/6% trifluoroacetic acid (TFA) containing 200 mM NaCl could be used as washing buffer. The elution of phosphopeptides is typically performed using alkaline solution such as 10% ammonia solution. For the application mode of Ti⁴⁺-IMAC, Ti⁴⁺-IMAC material could be packed into chromatographic column or small tip for enrichment of phosphopeptides, could also be used directly in bulk beads mode, Ti⁴⁺-IMAC beads and solution could be separated by centrifugation.

In this invention, we exemplified two examples using polymer material and mesoporous molecule sieve for preparation of Ti⁴⁺-IMAC materials, however this invention isn't limited to the above two materials. We further demonstrated the isolation and enrichment of phosphopeptides by Ti⁴⁺-IMAC based on polymer and mesoporous molecule sieve materials. In the following embodiments, all solution was prepared as volume ratio.

### EXAMPLE 1

### Preparation of Ti⁴⁺-IMAC Based on Polymer

Polymer containing phosphate group was prepared by free-radical co-polymerization of functional monomer containing phosphate group and cross-linker. Subsequently the obtained polymer was incubated with titanium ion solution to obtain the Ti⁴⁺-IMAC, which was further applied to isolate and enrich phosphopeptides. The first step to prepare such polymer is to prepare polymer reaction solution. Functional monomers and cross-linkers are the main components and form polymer framework after polymerization reaction. To adjust the polymer's chemical and physical properties, various monomers can also be used, wherein, at least one monomer containing phosphate group. Porous material has large surface area resulting in high adsorption capacity. In order to prepare porous polymer, porogenic solvents, which is organic solvent and don't take part in polymerization reaction, are added in the reaction solution. Pore will be formed due to spaces occupied by the porogenic solvents after polymerization reaction. The pore size and mechanic intensity of the polymer could be conveniently adjusted by optimizing the percentage of porogenic solvents in the reaction solution. Moreover, a few initiators are added to initiate the polymerization reaction. After preparation of the polymer containing phosphate group, the Ti⁴⁺-IMAC was obtained by incubating the polymer with titanium ion solution.

In this example, polymer containing phosphate group is obtained by direct copolymerization of a EGMP and BAA in trinary porogenic solvent consisting dimethylsulfoxide (DMSO), dodecanol and N,N′-dimethylformamide (DMF). The good hydrophilicity, stability, durability as well as alkali and acidic resistance of the prepared polymer material make it very suitable for use in phosphopeptide enrichment, where high concentrations of strong acids are often needed in the loading and washing buffers and extremely high pH is typically used to elute the trapped phosphopeptides. The polymer was then reacted with titanium ion solution such as titanium sulphate resulting in immobilization of titanium affinity chromatography material due to the strong chelating interaction between titanium ion and phosphate group on the polymer surface as shown in the FIG. 1a. The procedure for the preparation of Ti⁴⁺-IMAC is very convenient and simple. The specific enrichment of phosphopeptides from complex peptide mixtures were realized by the strong interaction between the immobilized titanium ions and phosphate group on the phosphopeptides. The scheme for isolation and enrichment of phosphopeptides using the Ti⁴⁺-IMAC was shown in FIG. 1b.

### (1) Preparation of Polymer, Affinity Material, Containing Phosphate Group

The polymer monolith containing phosphate group is prepared using the EGMP as functional monomer and BAA as cross linker. Briefly, the reaction mixture consisting of EGMP (13%, w/w), BAA, porogenic reagents (77%, w/w) including dimethylsulfoxide (540 μL), dodecanol (400 μL), N,N′-dimethylformamide (100 μL), and initiator reagents of Azodiisobutyronitrile (AIBN) (1%, w/w) is placed in a tube and sonicated for 20 min, and then purged with nitrogen for 10 min. After sealing, the tube was submerged into a water bath and allowed to react for 12 h at 60° C. After polymerization, a porous polymer monolith is obtained. Subsequently the obtained porous polymer monolith is ground into the irregular beads. The polymer beads are extensively washed with methanol for several times to remove the porogenic reagents, residue compounds. And then the polymer beads are collected by centrifuged at 20 000 g for 2 min and they are dried in vacuum for further usage.

### (2) Preparation of Ti⁴⁺-IMAC

The immobilized Ti⁴⁺ polymer beads (Ti⁴⁺-IMAC) were obtained by incubation of 10 mg polymer beads in 100 mM Ti(SO₄)₂ solution at room temperature overnight under gentle stirring. The obtained Ti⁴⁺-IMAC beads were centrifuged at 20 000 g for 2 min. After supernatant was removed, distilled water was used to wash the Ti⁴⁺-IMAC beads several times to remove the residue of titanium ion. The obtained Ti⁴⁺-IMAC beads were dispersed in 30% acetonitrile (ACN) containing 0.1% TFA solution before usage. The Ti⁴⁺-IMAC was applied to isolate and enrich phosphopeptides as shown in the example.3.

### EXAMPLE 2

### Preparation of Ti⁴⁺-IMAC Based on Inorganic Affinity Material

MCM-41 is a type of inorganic mesoporous molecule sieve, which is synthesized by hydrothermal self-organized method interacting by both organic leading compounds (surfactant) and organic species (monomer or oligomer) to form hexagonal crystal mesoporous molecule sieves. After removing the organic leading compounds by heat treatment or chemical methods, material called MCM-41 mesoporous molecule sieve obtained. It has hierarchically organized structure such as nanometer, micrometer and micrometer level. MCM-41 in nanometer level has honeycomb porous structure that of one dimension linear porous or hierarchically packed array and porous diameter from 1.5 nm to 30 nm. Furthermore, MCM-41, has very high surface area even up to 1200 m²/g with the typical 4 nm porous diameter. MCM-41 mesoporous molecule sieves have many advantages such as uniform and tunable mesoporous diameter, stable framework, easily modified unformed framework and inner surface area. Nowadays MCM-41 has widely been used as catalyzer, sorbent and catalyzer carrier by deriving of MCM-41. Also it is still a research hot for deriving of MCM-41 to satisfy various needs.

In this case, MCM-41 is synthesized as reported in previous work (Refers to reference 9) by hydrothermal method. The obtained MCM-41 is in turn derived to introduce silicon hydroxyl group, amido group and phosphate group by chemical reactions. Ti⁴⁺-IMAC material based on inorganic MCM-41 is obtained by incubation of the resulting MCM-41 material containing phosphate group in solution containing titanium ion. Phosphopeptides from complex peptide mixtures are isolated and enriched due to the strong interaction between titanium ion immobilized inorganic MCM-41 material and phosphate group on phosphopeptides as shown in the FIG. 1b.

### (1) Preparation of Inorganic MCM-41 Material Containing Phosphate Group.

### Preparation of Inorganic MCM-41 Material

Preparation of MCM-41 material is followed with previous method (refers to reference 9). Briefly, 2 g of hexadecyl trimethyl ammonium bromide is dissolved in 205 mL of ammonia (25% w/w) and 270 mL of ddH₂O. After stirring under heating, tetraethoxysilane (10 mL) was immediately added to homogenous solution reacting for 2 h. The resulting product is filtered and further washed with ddH₂O. The obtained product is dried under vacuum. The dried product is placed in a solution prepared with 450 mL of ethanol and 6 mL of hydrochloric acid (Analytical grade) for reaction by stirring at room temperature for 6 h. The obtained product is washed with ethanol and ddH₂O. The obtained organic MCM-41 is dried in vacuum at 120° C. The dimensions of the prepared MCM-41 mesoporous molecule sieves were with particle size of ca. 600 nm and pore size of ca. 3 nm.

### Activation of Inorganic MCM-41 Material

1 g of MCM-41 is dispersed in 20 mL of 6 M hydrochloric acid with gentle stirring for 5 h. The obtained product is thoroughly washed with ddH₂O for several times until pH at 7.0. The obtained product is washed with ethanol, and the product is dried in vacuum at 120° C.

### Animation of Inorganic MCM-41 Material

The dried MCM-41 (0.5 g) is placed in 25 mL toluene under Ar and subsequently 1.5 mL 3-aminopropyl-triethoxysilane is firstly added at room temperature for reaction of 5 h with stirring, and subsequently heating up 110° C. for refluxing 16 h. Then the toluene is removed by filtering and the obtained product is washed with toluene and ethanol for several times. The obtained product is dried in vacuum at 110° C.

Derivatization of Inorganic MCM-41 Material with Phosphate Groups

The dried MCM-41 (0.2 g) is placed in 15 mL toluene and subsequently 0.5 mL pyridine and 0.5 mL POCl₃ are added at room temperature for reaction of 18 h under gentle stirring, and subsequently heating up to 110° C. for refluxing of 16 h. Then the toluene is removed and the obtained product is washed with toluene and ddH₂O for several times. The obtained product is incubated in triethylamine for 20 min. Then the obtained product is washed with ddH₂O and ethanol, and dried in vacuum at 60° C.

### (2) Preparation of Ti⁴⁺-IMAC Based on Inorganic MCM-41 Material

The immobilization of Ti⁴⁺ is achieved by incubation of 10 mg phosphate group modified MCM-41 from the step of derivatization of inorganic MCM-41 material with phosphate groups referenced above in 100 mM Ti(SO₄)₂ solution at room temperature overnight under gentle stirring. The obtained Ti⁴⁺-IMAC material is centrifuged at 20 000 g for 2 min. After supernatant is removed, the Ti⁴⁺-IMAC material is washed by distilled water for several times to remove the residue of titanium ion. The obtained Ti⁴⁺-IMAC material is dispersed in 30% ACN containing 0.1% TFA solution before usage. The Example 3.2 demonstrated the isolation and enrichment of phosphopeptides by Ti⁴⁺-IMAC based on MCM-41 inorganic affinity material.

### EXAMPLE 3

### Selective Enrichment of Phosphopeptides Using the Ti⁴⁺-IMAC

### Preparation of Peptide Samples

α-Casein and β-casein (1 mg) are dissolved in 1 mL ammonium bicarbonate (50 mM, pH 8.2) and digested for 16 h at 37° C. with trypsin at enzyme-to-protein ratio of 1:40 (w/w). BSA (6.6 mg) and ovalbumin (4.5 mg) are respectively dissolved in 1 mL denaturing buffer containing 8 M urea and 50 mM ammonium bicarbonate and incubated for 3 h. To the obtained protein solution 20 μL of 50 mM DTT is added and the solution is incubated for 2 h at 37° C. And then 40 μL of 50 mM IAA is added and the obtained solution is incubated for an additional 30 min at room temperature in dark. After that, the mixture is diluted with 50 mM ammonium bicarbonate by 10 folds and incubated for 16 h at 37° C. with trypsin at an enzyme/substrate ratio of 1:40 (w/w) to produce proteolytic digests. The obtained tryptic digest of α-casein, β-casein and ovalbumin are kept in the freezer under −30° C. for further usage. The obtained peptide solution of BSA is lyophilized by a vacuum concentrator for usage.

### EXAMPLE 3.1

### Enrichment of Phosphopeptides Using Ti⁴⁺-IMAC Based on Polymer Beads

### (1) Sample 1:

1 μL tryptic digest of α-casein is prepared in loading buffer of 80% ACN/6% TFA, and the peptide mixture is firstly incubated with 5 μL of Ti⁴⁺-IMAC beads (10 mg.mL⁻¹) in loading buffer with vibration for 30 min at room temperature. Supernatant is removed after centrifugation. And beads with captured phosphopeptides are in turn washed with 30 μL solutions of 200 mM NaCl, 80% ACN/6% TFA and 30% ACN/0.1% TFA, respectively. The bound phosphopeptides are then eluted with 10 μL of 10% NH₃.H₂O under the sonication for 10 min. After centrifugation at 20 000 g for 5 min, the supernatant is collected and lyophilized to dryness. 2 μL DHB solution (25 mg/mL in 70% ACN) containing 1% H₃PO₄ (v/v) was added to redissolve the enriched phosphopeptides, and 0.5 μL of resulting solution was deposited on the MALDI target for MALDI-TOF MS analysis. MALDI-TOF mass spectrometry analysis was performed on a Bruker Autoflex time-of-flight mass spectrometer (Bruker, Bermen, Germany). The instrument was equipped with a delayed ion-extraction device and a pulsed nitrogen laser operated at 337 nm and its available accelerating potentials is in the range of ±20 kV. All mass spectra reported were obtained in the positive ion linear mode with delayed extraction for 90 ns. External mass calibration was obtained by using angiotensinII and insulin B that bracketed the mass range of interest. And each mass spectrum was typically summed with 30 laser shots.

### Analytical Results

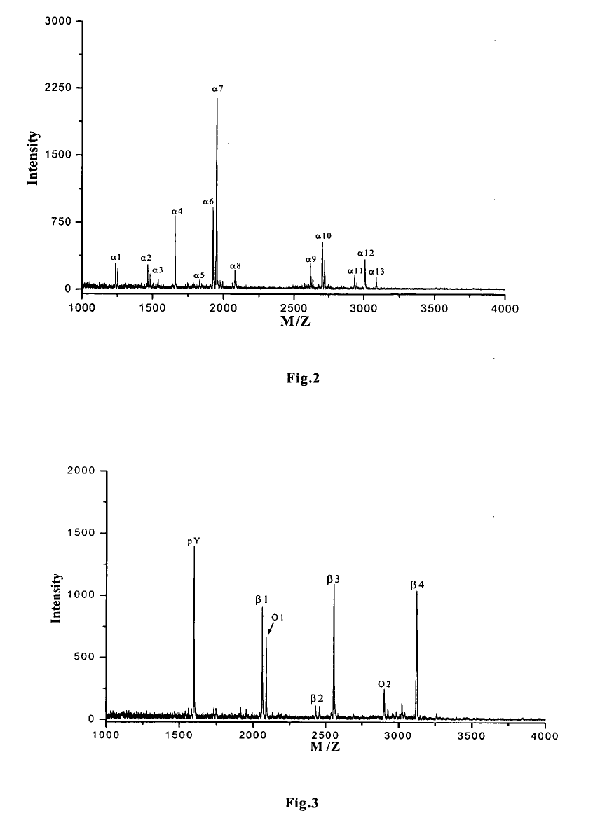

As shown in the FIG. 2, the thirteen phosphopeptides from tryptic digest of α-casein are specifically isolated and enriched by Ti⁴⁺-IMAC, and detected by MALDI-TOF MS.

### (2) Sample 2:

Peptide Mixtures Containing Peptides Originating from Tryptic Digests of β-Casein, Ovalbumin and a Model Tyrosine Phosphorylated Peptide

Peptide mixture containing peptides originating from 1 pmol of tryptic digests of β-casein, ovalbumin, BSA and 1 pmol of model tyrosine phosphorylated peptides (RRLIEDAEpYAARG, MW 1599.00) is dissolved in 80% CAN/ 6% TFA, and the peptide mixtures were firstly incubated with 5 μL of Ti⁴⁺-IMAC beads (10 mg.mL¹) in loading buffer with vibration at room temperature for 30 min. Supernatant was removed after centrifugation. And beads with captured phosphopeptides were in turn washed with 30 μL solutions of 200 mM NaCl/50% ACN/6% TFA and 30% ACN/0.1% TFA, respectively. The bound phosphopeptides were then eluted with 10 μL of 10% NH₃.H₂O under the sonication for 10 min. After centrifugation at 20 000 g for 5 min, the supernatant was collected and lyophilized to dryness. 2 μL DHB solution (25 mg/mL in 70% ACN) containing 1% H₃PO₄ (v/v) was added to redissolve the enriched phosphopeptides, and 0.5 μL of resulting solution was deposited on the MALDI target for MALDI-TOF MS analysis.

### Analytical Results

As shown in the FIG. 3, the seven phosphopeptides from tryptic digest of β-casein, ovalbumin and the model tyrosine phosphorylated peptide were specifically isolated and enriched by Ti⁴⁺-IMAC, and detected by MALDI-TOF MS. The tyrosine phosphorylated peptide can be equally enriched. And DTT, IAA and urea introduced during proteolytic digestion and glycopeptides in tryptic digest of ovalbumin do not interfere with the isolation and enrichment of phosphopeptides.

### (3) Sample 3:

### Semi-Complex Peptide Mixtures

Peptide mixtures originating from a tryptic digest of 1 μL of the α-casein (1 pmol.μL⁻¹) and 1 μL, 5 μL of tryptic digest of non-phosphoprotein of BSA (100 pmol.μL⁻¹ or 500 pmol.μL⁻¹) were dissolved in 80% ACN/6% TFA and then incubated with 5 μL of Ti⁴⁺-IMAC beads (10 mg.mL⁻¹) in loading buffer with vibration of 30 min at room temperature. Supernatant was removed after centrifugation. And the beads with captured phosphopeptides were in turn washed with 30 μL of 200 mM NaCl/50% ACN/6% TFA and 30% ACN/0.1% TFA, respectively. The bound phosphopeptides were then eluted with 10 μL of 10% NH₃.H₂O under the sonication for 10 min. After centrifugation at 20 000 g for 5 min, the supernatant was collected and lyophilized to dryness. 2 μL DHB solution (25 mg/mL in 70% ACN) containing 1% H₃PO₄ (v/v) was added to redissolve the enriched phosphopeptides, and 0.5 μL of resulting solution was deposited on the MALDI target for MALDI-TOF MS analysis.

### Analytical Results

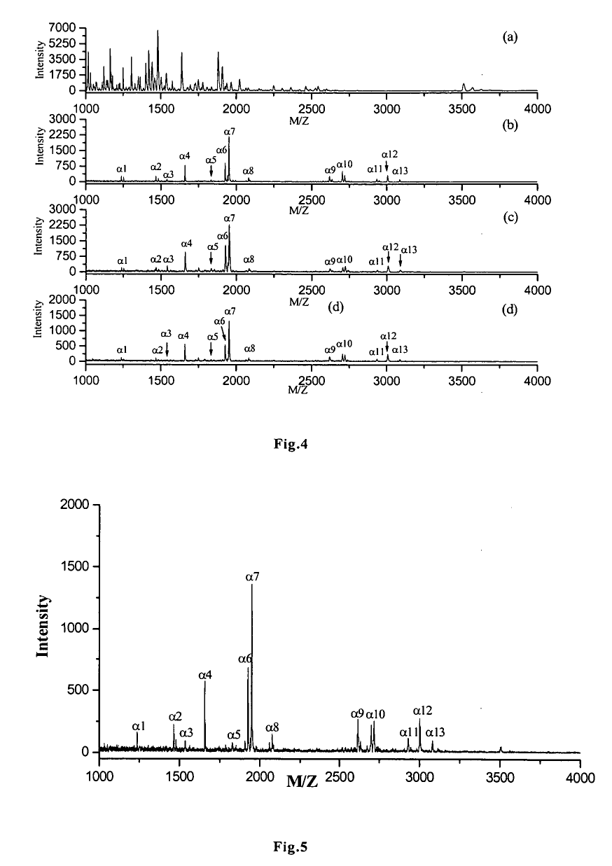

Seeing FIG. 4, wherein FIG. 4a shows that many non-phosphopeptides peaks dominate the mass spectrum when the tryptic digests of α-casein and BSA with molar ratio of 1:100 was directly analyzed without enrichment. However, it can be seen from (c) and (d), thirteen phosphopeptides clearly presented and non-phosphopeptides were hardly observed in the MALDI mass spectrum even with 100-fold or 500-fold dilution by tryptic digests of non-phosphoprotein of BSA after Ti⁴⁺-IMAC enrichment. For comparison, the MALDI spectra for analysis of phosphopeptides enriched from single tryptic digest of α-casein by Ti⁴⁺-IMAC is shown in (b).

### EXAMPLE 3.2

### Enrichment of Phosphopeptides Using Ti⁴⁺-IMAC Based on Inorganic MCM-41 Material

### (1) Sample 1:

Tryptic digest of 1 μL α-casein (1 pmol.μL⁻¹) was prepared in loading buffer of 80% ACN/6% TFA obtaining peptide mixture. The peptide mixture was firstly incubated with 5 μL of MCM-41 material with immobilized titanium ion (10 mg.mL⁻¹) in loading buffer with vibration of 30 min at room temperature. Supernatant was removed after centrifugation. And the MCM-41 material with captured phosphopeptides were in turn washed with 30 μL of 200 mM NaCl/80% ACN/6% TFA and 30% ACN/0.1% TFA, respectively, for 10 min. The bound phosphopeptides were then eluted with 10 μL of 10% NH₃.H₂O under the sonication for 10 min. After centrifugation at 20 000 g for 5 min, the supernatant was collected and lyophilized to dryness. 2 μL DHB solution (25 mg/mL in 70% ACN) containing 1% H₃PO₄ (v/v) was added to redissolve the enriched phosphopeptides, and 0.5 μL of resulting solution was deposited on the MALDI target for MALDI-TOF MS analysis.

### Analytical Results

As shown in FIG. 5, thirteen phosphopeptides from tryptic digest of α-casein can be specifically isolated and enriched by Ti⁴⁺-IMAC based on inorganic MCM-41 material, and detected by MALDI-TOF MS.

### (2) Sample 2:

Peptide Mixtures Contained Peptides Originating from a Tryptic Digests of β-Casein and a Model Tyrosine Phosphorylated Peptide

1 μL peptide mixture containing peptides originating from 1 pmol of tryptic digests of β-casein and model tyrosine phosphorylated peptide (RRLIEDAEpYAARG, MW1599.00) was dissolved in 80% ACN/6% TFA obtaining peptide mixture, and the peptide mixture was firstly incubated with 5 μL of MCM-41 material with immobilized titanium ion (10 mg.mL¹) in loading buffer with vibration of 30 min at room temperature. Supernatant was removed after centrifugation. And the MCM-41 material with captured phosphopeptides were in turn washed with 30 μL of 200 mM NaCl/50% ACN/6% TFA and 30% ACN/0.1% TFA, respectively, for 10 min. The bound phosphopeptides were then eluted with 10 μL of 10% NH₃.H₂O under the sonication for 10 min. After centrifugation at 20 000 g for 5 min, the supernatant was collected and lyophilized to dryness. 2 μL DHB solution (25 mg/mL in 70% ACN) containing 1% H₃PO₄ (v/v) was added to redissolve the enriched phosphopeptides, and 0.5 μL of resulting solution was deposited on the MALDI target for MALDI-TOF MS analysis.

### Analytical Results

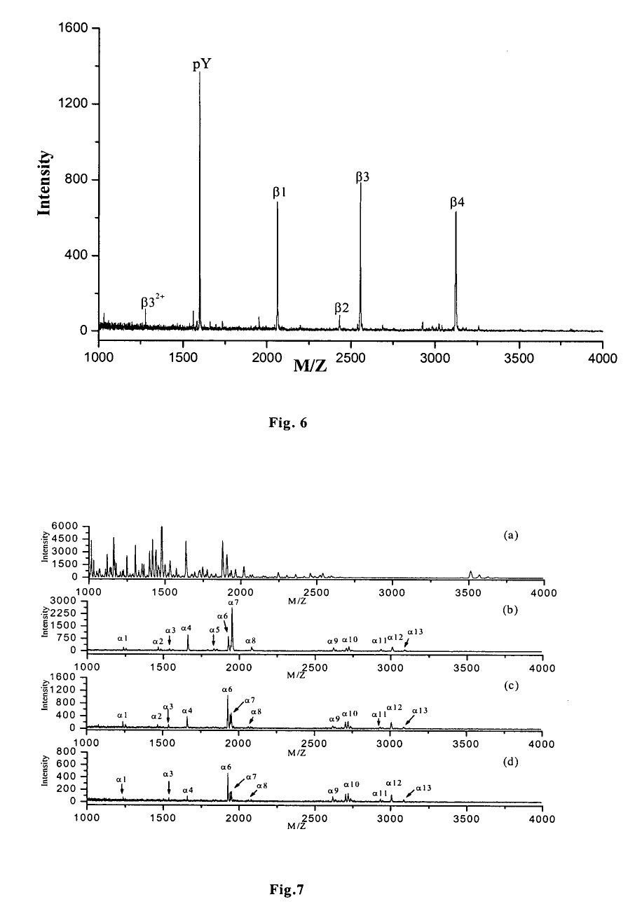

As shown in FIG. 6, five phosphopeptides from tryptic digest of β-casein, ovalbumin and a model tyrosine phosphorylated peptide were specifically isolated and enriched by Ti⁴⁺-IMAC based on inorganic MCM-41 material, and detected by MALDI-TOF MS. Meanwhile, the tyrosine phosphorylated peptide can be equally enriched.

### COMPARATIVE EXAMPLE 1

### Comparing with Immobilized Zirconium Metal Affinity Chromatographic (Zr⁴⁺-IMAC) Beads

### Comparative Example 1-1 Polymer Material as Affinity Media

### (1) Preparation of Immobilized Zirconium Ion Polymer Affinity Material

The polymer material containing phosphate group for the immobilization of zirconium ion is the same with that in the example 3.1. The immobilization of zirconium ion were obtained by incubation of 10 mg polymer beads in 10 mL, 100 mM ZrOCl₂ solution at room temperature overnight under gentle stirring. The obtained Zr⁴⁺-IMAC beads were centrifuged at 20 000 g for 2 min. After supernatant was removed, distilled water was used to wash the Zr⁴⁺-IMAC beads for several times to remove the residue of zirconium ion. The obtained Zr⁴⁺-IMAC was dispersed in 30% ACN containing 0.1% TFA solution before usage.

(2) Isolate and Enrich Phosphopeptides from Semi-Complex Peptide Mixture Using the Zr⁴⁺-IMAC

Peptide mixtures originating from a tryptic digest of 1 μL of the α-casein (1 pmol.μL⁻¹) and 1 μL, 5 μL of tryptic digest non-phosphoprotein of BSA (100 pmol.μL⁻¹ or 500 pmol.μL⁻¹) were dissolved in 80% ACN/6% TFA and then incubated with 5 μL of Zr⁴⁺-IMAC beads (10 mg.mL¹) in loading buffer with vibration of 30 min at room temperature. Supernatant was removed after centrifugation. And the beads with captured phosphopeptides were in turn washed with 30 μL of 200 mM NaCl/80% ACN/6% TFA and 30% ACN/0.1% TFA for 10 min, respectively. The bound phosphopeptides were then eluted with 10 μL of 10% NH₃.H₂O under the sonication for 10 min. After centrifugation at 20 000 g for 5 min, the supernatant was collected and lyophilized to dryness. 2 μL DHB solution (25 mg/mL in 70% ACN) containing 1% H₃PO₄ (v/v) was added to redissolve the enriched phosphopeptides, and 0.5 μL of resulting solution was deposited on the MALDI target for MALDI-TOF MS analysis.

### Analytical Results

As shown in the FIG. 7, wherein (a), many non-phosphopeptides dominated the MALDI spectrum obtained for direct analysis of tryptic digests of α-casein (1 pmol) and BSA (100 pmol). (b) shown that the thirteen phosphopeptides peaks enriched from tryptic digest of α-casein by Zr⁴⁺-IMAC. As shown in the FIGS. 7c and d, thirteen and twelve phosphopeptides were enriched by Zr⁴⁺-IMAC in the presence of 100-folder and 500-folder dilution with tryptic digest of BSA, respectively. The non-phosphopeptides was hardly retained from (c) and (d). Although the Zr⁴⁺-IMAC shows high specificity, the enrichment preference was observed between Ti⁴⁺-IMAC and Zr⁴⁺-IMAC. As shown in (c) and (d), singly phosphorylated peptide (α6, YKVPQLEIVPNpSAEER) is the dominant peak following with Ti⁴⁺-IMAC enrichment. However the doubly phosphorylated peptide (α7, DIGpSEpSTEDQAMEDIK) is the dominant peak for Ti⁴⁺-IMAC. So the Ti⁴⁺-IMAC shows more strong enrichment preference toward singly phosphorylated peptides, and Zr⁴⁺-IMAC shows more strong enrichment preference toward doubly phosphorylated peptides.

### Comparative Example 1-2 GMA-EDMA Beads as Affinity Media

### (1) Preparation of Zr⁴⁺-IMAC Based on GMA-EDMA Beads

In this case, the GMA-EDMA beads used was prepared as reported in previous work (19. Feng, S. et al, Mol. Cell. Proteomics, P1656-1665, (2007)).

(2) Isolation of Phosphopeptides from Semi-Complex Peptide Mixtures Using Zr⁴⁺-IMAC Based on GMA-EDMA Beads

1 μL peptide mixtures originating from a tryptic digest of 1 μL of the α-casein (1 pmol.μL⁻¹) and 1 μL, 5 μL of tryptic digest non-phosphoprotein of BSA (100 pmol.μL¹ or 500 pmol.μL⁻¹) were dissolved in 80% ACN/6% TFA and then incubated with 5 μL of Zr⁴⁺-IMAC beads based on GMA-EDMA (10 mg.mL¹ in 30% ACN/0.1% TFA) in loading buffer with vibration of 30 min. Supernatant was removed after centrifugation. And the beads with captured phosphopeptides were in turn washed with 30 μL of 200 mM NaCl/50% ACN/6% TFA and 30% ACN/0.1% TFA, respectively. The bound phosphopeptides were then eluted with 10 μL of 10% NH₃.H₂O under the sonication for 10 min. After centrifugation at 20 000 g for 5 min, the supernatant was collected and lyophilized to dryness. 2 μL DHB solution (25 mg/mL in 70% ACN) containing 1% H₃PO₄ (v/v) was added to redissolve the enriched phosphopeptides, and 0.5 μL of resulting solution was deposited on the MALDI target for MALDI-TOF MS analysis.

### Analytical Results

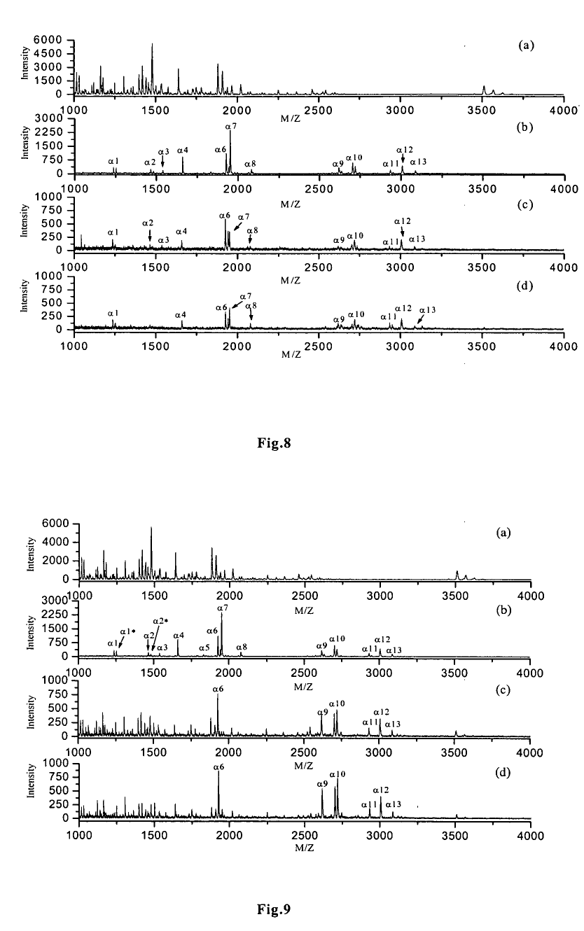

As shown in the FIG. 8, wherein (a), many non-phosphopeptides dominated the MALDI spectrum obtained for direct analysis of tryptic digests of α-casein (1 pmol) and BSA (100 pmol). (b) shown that thirteen phosphopeptides can be enriched from tryptic digest of α-casein by Zr⁴⁺-IMAC. (c) demonstrated that thirteen phosphopeptides can be specifically enriched from tryptic digest of α-casein in the presence of 100-fold dilution of BSA digest by Zr⁴⁺-IMAC. The FIG. 8d demonstrated that ten phosphopeptides enriched from tryptic digest of α-casein in the presence of 500-fold dilution of BSA digest without non-phosphopeptide peak and the phosphopeptide signal intensity was obviously lower than that with 100-fold contamination with BSA digest. Although Zr⁴⁺-IMAC based on GMA-EDMA also shows high specificity, the limited phosphate group obtained by incompleted chemical reaction and further resulted in much less binding sites to phosphopeptides. So, the signal intensity of phosphopeptides was one-fold lower than Ti⁴⁺-IMAC based on polymer as shown in (c) and (d) and FIG. 4, wherein (c) and (d). Also, the Zr⁴⁺-IMAC showed the enrichment preference toward multiply phosphorylated peptide and Ti⁴⁺-IMAC showed the enrichment preference toward singly phosphorylated peptide.

### COMPARATIVE EXAMPLE 2

### Comparing with Immobilized Fe³⁺ Affinity Chromatography (Fe³⁺-IMAC) for Isolation and Enrichment of Phosphopeptides

### (1) Activation of Fe³⁺-IMAC

Fe³⁺-IMAC beads (POROS 20 MC beads) were activated according to the manufacture's instructions. 10 mg beads were washed with 50 mM EDTA and 1 M NaCl for two times and then ddH₂O for three times. The washed beads were placed in the 100 mM FeCl₃ for incubation with 30 min for three times. The obtained Fe³⁺-IMAC beads was in turn washed with 0.1% HAC, 500 mM NaCl and ddH₂O, and then dispersed in 1 mL of ddH₂O for usage.

(2) Isolation of Phosphopeptides from Semi-Complex Peptide Mixtures Using Fe³⁺-IMAC

The Fe³⁺-IMAC was equilibrated with the loading buffer of 50% ACN/0.1% TFA for 30 min and the solution was removed by centrifugation. Obtained Fe³⁺-IMAC beads was incubated with semi-complex peptide mixture of tryptic digests of α-casein (1 pmol) and BSA (100 pmol or 500 pmol) dissolved in 50% ACN/0.1% TFA for 30 min with vibration at room temperature. The beads were in turn washed with 100 μL of 50% ACN/0.1% TFA; 75% ACN/10% CH₃OH/10% HAC and 10% HAC. Lastly, the bound phosphopeptides were eluted by 10 μL of NH₃.H₂O (10%). After centrifugation at 20 000 g for 5 min, supernatant was collected and lyophilized to dryness. 2 μL DHB solution (25 mg/mL in 70% ACN) containing 1% H₃PO₄ (v/v) was added to redissolve the enriched phosphopeptides, and 0.5 μL of resulting solution was deposited on the MALDI target for MALDI-TOF MS analysis.

### Analytical Results

As shown in FIG. 9, wherein (a), many non-phosphopeptide peaks dominated the MALDI spectrum obtained for direct analysis of tryptic digests of α-casein (1 pmol) and BSA (100 pmol). (b) demonstrated that thirteen phosphopeptides could be enriched from tryptic digest of α-casein by Fe3+-IMAC. As shown in (c) and (d), only five multiply phosphorylated peptides were isolated and enriched in the presence of 100-fold and 500-fold contamination of BSA digest with many non-phosphopeptides presented. So, the Fe³⁺-IMAC has poorer specificity than Ti⁴⁺-IMAC or Zr⁴⁺-IMAC.

### COMPARATIVE EXAMPLE 3

### Comparing with TiO2 for Isolation and Enrichment of Phosphopeptides from Semi-Complex Peptide Mixtures

Peptide mixtures originating from a tryptic digest of 1 μL of the α-casein (1 pmol.μL⁻¹) and 1 μL, 5 μL of tryptic digest of non-phosphoprotein of BSA (100 pmol.μL⁻¹ or 500 pmol.μL⁻¹) were dissolved in 80% ACN/6% TFA and then incubated with 5 μL of TiO₂ beads (GL sciences Inc. Tokyo, Japan) (10 mg.mL⁻¹ in 30% ACN/0.1% TFA) in loading buffer with vibration of 30 min at room temperature. Supernatant was removed after centrifugation. And the beads with captured phosphopeptides were in turn washed with 30 μL of 200 mM NaCl/50% ACN/6% TFA and 30% ACN/0.1% TFA for 10 min. The bound phosphopeptides were then eluted with 10 μL of 10% NH₃.H₂O under the sonication for 10 min. After centrifugation at 20 000 g for 5 min, the supernatant was collected and lyophilized to dryness. 2 μL DHB solution (25 mg/mL in 70% ACN) containing 1% H₃PO₄ (v/v) was added to redissolve the enriched phosphopeptides, and 0.5 μL of resulting solution was deposited on the MALDI target for MALDI-TOF MS analysis.

### Analytical Results

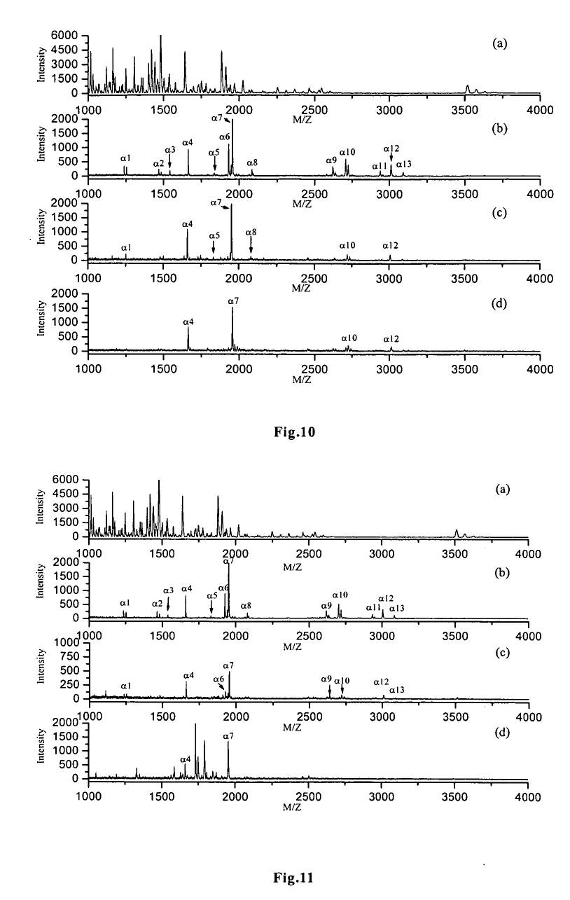

As shown in FIG. 10, wherein, (a) many non-phosphopeptides dominated the MALDI spectrum obtained for direct analysis of tryptic digests of α-casein (1 pmol) and BSA (100 pmol). (b) demonstrated the thirteen phosphopeptides enriched only from tryptic digest of α-casein by TiO₂ beads. As shown in (c) and (d), the seven and four phosphorylated peptides were isolated and enriched by TiO₂ beads and clearly presented in the presence of 100-fold and 500-fold contamination of BSA digest, respectively. However, the peak intensity of phosphopeptides was obviously lower than only simple α-casein digests enriched. So, the specificity and capacity of TiO₂ enrichment is obviously lower than using the Ti⁴⁺-IMAC or Zr⁴⁺-IMAC enrichment.

### COMPARATIVE EXAMPLE 4

### Comparing with ZrO₂ for Isolation and Enrichment of Phosphopeptides from Semi-Complex Peptide Mixtures

Preparation of ZrO₂ and enrichment of phosphopeptides using ZrO₂ were followed with the previous work (refers to 13, Zhou, H. et al, “Highly specific enrichment of phosphopeptides by zirconium dioxide nanoparticles for phosphoproteome analysis”, Electrophoresis, P2201-2215, (2007)). Peptide mixtures originating from a tryptic digest of 1 μL of the α-casein (1 pmol.μL⁻¹) and 1 μL or 5 μL of tryptic digest non-phosphoprotein of BSA (100 pmol.μL⁻¹ or 500 pmol.μL⁻¹) were dissolved in 50% ACN/10% HAC and then incubated with 5 μL of ZrO₂ beads (10 mg.mL⁻¹ in 50% ACN/10% HAC) in loading buffer with vibration of 30 min at room temperature. Supernatant was removed after centrifugation. And the beads with captured phosphopeptides were in turn washed with 30 μL of 50% ACN/10% HAC and 50% ACN with vibration for 10 min. The bound phosphopeptides were then eluted with 10 μL of 10% NH₃.H₂O under the sonication for 10 min. After centrifugation at 20 000 g for 5 min, the supernatant was collected and lyophilized to dryness. 2 μL DHB solution (25 mg/mL in 70% ACN) containing 1% H₃PO₄ (v/v) was added to redissolve the enriched phosphopeptides, and 0.5 μL of resulting solution was deposited on the MALDI target for MALDI-TOF MS analysis.

### Analytical Results

As shown in the FIG. 11, wherein (a) many non-phosphopeptide peaks dominated the MALDI spectrum for direct analysis of tryptic digests of α-casein (1 pmol) and BSA (100 pmol). (b) demonstrated the thirteen phosphopeptides enriched only from tryptic digest of α-casein by ZrO₂. The eight phosphopeptides can be specifically isolated and enriched from tryptic digests of α-casein (1 pmol) and BSA (100 pmol) as shown in (c). The two singly phosphorylated peptides were clearly presented in the presence of contamination of 500-fold BSA digests. Meanwhile, a few non-phosphopeptides were also retained as shown in (d). So, the specificity and capacity using ZrO₂ enrichment is obviously lower than Ti⁴⁺-IMAC and Zr⁴⁺-IMAC enrichment.

TABLE 1

Overview molecular weight, peptide sequence and phosphory-
lation sites of observed phosphopeptides derived from
tryptic digests of α-casein, β-casein and ovalbumin and
standard tyrosine phosphorylated peptides. The lowercase
‘p’ represents a phosphate group.

| | | | |
|---|---|---|---|
| | | Phosphory- | |
| | | lation | |
| No | [M + H]⁺ | sites | Peptide sequence |

| | | | | |
|---|---|---|---|---|
| α-casein | α1 | 1237.50 | 1 | TVDMEpSTEVF |
| | α2 | 1466.61 | 1 | TVDMEpSTEVFTK |
| | α3 | 1538.59 | 2 | EQLpSTpSEENSKK |
| | α4 | 1660.79 | 1 | VPQLEIVPNpSAEER |
| | α5 | 1832.83 | 1 | YLGEYLIVPNpSAEER |
| | α6 | 1927.69 | 2 | DIGpSEpSTEDQAMEDIK |
| | α7 | 1951.95 | 1 | YKVPQLEIVPNpSAEER |
| | α8 | 2080.04 | 1 | KKYKVPQLEIVPNpSAEERL |
| | α9 | 2618.72 | 4 | NTMEHVpSpSpSEESIIpSQETYK |
| | α10 | 2719.90 | 5 | QMEAEpSIpSpSpSEEIVPNPNpSVEQK |
| | α11 | 2935.15 | 3 | EKVNELpSKDIGpSEpSTEDQAMEDIKQ |
| | α12 | 3008.01 | 4 | NANEEEYSIGpSpSpSEEpSAEVATEEVK |
| | α13 | 3087.99 | 5 | NANEEEYpSIGpSpSpSEEpSAEVATEEVK |
| β-casein | β1 | 2061.83 | 1 | FQpSEEQQQTEDELQK |
| | β2 | 2431.03 | 1 | IEKFQpSEEQQQTEDELQDK |
| | β3 | 2556.09 | 1 | FQpSEEQQQTEDELQDKJHPF |
| | β4 | 3122.27 | 4 | RELEELNVPGEIVEpSLpSpSpSEESITR |
| Ovalbumin | O1 | 2090.45 | 1 | EVVGpSAEAGVDAASVSEEFR |
| | O2 | 2903.63 | 1 | FDKLPGFGDpSIEAQCGTSVNVHSSLR |
| pY | | 1599.00 | 1 | RRLIEDEpYAARG |

What is claimed:

1. An immobilized titanium ion affinity chromatography material is prepared by incubating solid support containing phosphate groups with titanium ions solution, and the titanium ions are immobilized on the solid support because of strong interaction between titanium ions and phosphate groups on the surface of the solid support, which resulted in the formation of immobilized titanium ion affinity chromatography material (Ti⁴⁺-IMAC).

2. The immobilized titanium ion affinity chromatography material of the claim 1, characterized in that the said solid support containing phosphate groups being covalently bound on the surface of the solid support with the structure as follows:

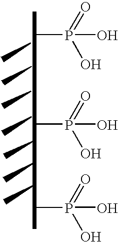

3. A method for preparation of the immobilized titanium ion affinity chromatography material of the claim 1, characterized in that the solid support containing phosphate group is incubated with titanium ion solution, and the titanium ion is immobilized on the surface of the solid support due to the strong interaction between the titanium ions and phosphate groups on the surface of the support, which resulted in the formation of immobilized titanium ion affinity chromatography material (Ti⁴⁺-IMAC).

4. The method for preparation of the claim 3, characterized in that titanium ion solution adopted the preparation is titanium sulphate solution.

5. The method for preparation of the claim 3, characterized in that the said affinity chromatography material is prepared by chemical derivatization of solid support to couple phosphate group on its surface.

6. The method of the claim 5, characterized in that the said solid support using any one selected from the group consisting of silicon gel particle, organic polymer beads, agarose beads, monolithic material, nanomaterial, mesoporous material, magnetic bead and chip material.

7. The method of the claim 3, characterized in that the said affinity chromatography material is obtained by polymerization of functional monomer containing phosphate group.

8. The method of the claim 7, characterized in that the said functional monomer is ethylene glycol methacrylate phosphate; cross-liner adopted the preparation is bis-acrylamide, ethylene dimethacrylate or divinylbenzene; and the said polymerization reaction is bulk polymerization, suspension polymerization, emulsion polymerization or solution polymerization.

9. An application of immobilized titanium ion affinity chromatography material (Ti⁴⁺-IMAC) of the claim 1, characterized in that Ti⁴⁺-IMAC is applied to isolate and enrich phosphopeptides; phosphopeptides are retained on Ti⁴⁺-IMAC because of the strong chelating interaction between the immobilized titanium ion and phosphopeptides resulting in specific isolation and enrichment of phosphopeptides from complex proteolytic digests.
